(19) United States
(12) Patent Application Publication
(10) Pub. No.: US 2025/0392821 A1
(43) Pub. Date: Dec. 25, 2025

(54) IMAGING DEVICE HAVING EXTENDED ZOOM FUNCTIONALITY AND FOCUS TRACKING

(71) Applicant: Karl Storz SE & Co. KG, Tuttlingen (DE)

(72) Inventors: Chang-Hae KIM, Tuttlingen (DE); Benedikt KÖHLER, Tuttlingen (DE); Stephan SCHRADER, Tuttlingen (DE); Valery TODA, Tuttlingen (DE)

(73) Assignee: Karl Storz SE & Co. KG, Tuttlingen (DE)

(21) Appl. No.: 18/880,344

(22) PCT Filed: Jun. 29, 2023
(86) PCT No.: PCT/EP2023/067941

§ 371 Date: Dec. 31, 2024

(30) Foreign Application Priority Data
Jul. 4, 2022 (DE) 10 2022 116 672.4

(51) Int. Cl. H04N 23/69; G03B 13/36; H04N 23/50; H04N 23/60; H04N 23/67

(52) U.S. Cl. CPC H04N 23/69; G03B 13/36; H04N 23/555; H04N 23/64; H04N 23/675

(57) ABSTRACT

An imaging device for the scalable visual depiction of a region to be observed includes an optical image capture apparatus, optical and/or electronic scaling means for scaling the captured image, and an adjustable focusing apparatus. A robotic holding arm moves the image capture apparatus relative to the region to be observed and provides a mechanical scaling function through adaptation of an axial distance (d) between the image capture apparatus and the region to be observed. An input unit captures a user-side input command for scaling the captured image on a display unit. A control apparatus sets the image scaling, the mechanical scaling function and sets the focusing apparatus. The control apparatus executes the setting of the mechanical scaling function and the focusing apparatus by actuating the holding arm and the focusing apparatus, based only on the input command detected by the input unit for scaling the captured image.

### CROSS-REFERENCE TO RELATED APPLICATIONS

This application is the U.S. national stage of PCT/EP2023/067941 filed on Jun. 29, 2023, which claims priority of German Patent Application No. DE 10 2022 116 672.4 filed on Jul. 4, 2022, the contents of which are incorporated herein.

### TECHNICAL FIELD

The present disclosure relates to an imaging device and to a method for the scalable visual depiction of a region to be observed, in particular an operation region, with an extended zoom functionality and optimized focus tracking.

### BACKGROUND

Endoscopes and exoscopes for use in diagnostic or surgical procedures on patients are well known in the art. The endoscope or exoscope is sometimes mounted on a motorized holding arm, which is positioned by the user in such a way that a camera unit of the endoscope or exoscope appropriately displays the area to be captured, usually a surgical site, for the operating surgeon. The captured image is then output as a live image on a suitable imaging device such as a screen. The distance between the holding arm and the patient is generally chosen in such a way that the image portion displayed is suitable for the surgeon, and at the same time good access to the patient is guaranteed. Once a suitable placement and orientation of the holding arm and the attached endoscope or exoscope has been found, the focus of the camera unit is usually adjusted manually.

Furthermore, it is state of the art that the corresponding camera unit of the endoscope or exoscope is equipped with scaling means, in particular an optical or electronic or digital zoom, with which the user can enlarge or reduce an image of the captured area as required before and/or during a treatment or procedure. In order to extend a zoom range, it is also already known to provide, in addition to scaling means of the camera unit, a mechanical scaling function by changing the holding arm position along an optical axis of an endoscope, and thus by changing a distance between the camera unit and the captured area.

However, re-adjusting or tracking the focus adjustment of the camera unit is a challenge, as the field depth is very small, especially with microscope lenses, so that even a small change in the position of the holding arm relative to the captured area or surgical site can lead to blurring. For example, providing continuous autofocus carries the risk of unwanted pulsating image focus or focusing on an unwanted subregion within the overall captured area.

Manual or user-triggered focus adjustment is also disadvantageous, since the user usually has both hands on the instruments at the site during a procedure or treatment, and additional manual operation of the device is undesirable.

U.S. Pat. No. 11,033,338 B2, for example, discloses an imaging system comprising an endoscope with a camera head for image capture and a display connected thereto for the enlarged depiction of a captured image area during an endoscopic procedure, wherein the endoscope is arranged on a movable holding arm. The system has a control apparatus for controlling the camera head on the endoscope and a control apparatus for controlling the holding arm, wherein the camera head is designed to provide an optical and electronic zoom. In addition to the optical and electronic scaling function of the camera head, a mechanical scaling function is provided by changing the holding arm position along an optical axis of the endoscope. The focus adjustment of the camera head is adjusted using an autofocusing device, which has the disadvantages described above.

U.S. Pat. No. 8,715,167 B1 discloses a telesurgical system for minimally invasive interventions, comprising an input device and a robot system connected thereto, having a first manipulator with an endoscope attached thereto for optical image acquisition, which is connected to an input console of the telesurgical system for image display, and a second manipulator which is designed to hold and guide a surgical instrument, and which is connected to an input device on the input console. The telesurgical system is designed to change a focus and a scaling factor in response to a movement of the robot system, for example to maintain focusing of the image capture apparatus when the distance to an object to be captured changes. The robot system comprises multiple sensors which monitor a movement of the robot system elements, determine a change in distance from an initial focus point in a coordinate system of the robot system and, based on this, adjust the focus setting of the image capture apparatus. On the one hand, this enables precise position detection and adjustment of the focus setting based on this, but on the other hand, this requires a large number of sensors on the robot system and associated information monitoring of each of these sensors, which not only results in increased hardware expenditure, but also an increased susceptibility to errors in the focus adjustment that is directly dependent on it.

### SUMMARY

The object of the present disclosure is therefore to overcome the above-mentioned disadvantages of the prior art at least partially. In particular, it is an object of the disclosure to provide an improved imaging device and a corresponding method which enables a scalable imaging of an operation region with extended zoom functionality while simultaneously providing a simple and effective tracking of the image sharpness of the image.

The object presented above is achieved by a device and method according to the embodiments disclosed herein.

Advantageous further developments of the disclosure are indicated in the dependent claims. All combinations of at least two of the features disclosed in the description, the claims and/or the figures fall within the scope of the disclosure. It is understood that exemplary embodiments and embodiments that are described with respect to the device according to claim 1 may relate in an equivalent, albeit not identically worded, form to the method according to claim 16 without being explicitly mentioned therefor. It is also understood that transformations that are customary in the language and/or a meaningful replacement of respective terms in the context of customary language practice, in particular the use of synonyms supported by generally recognized language literature, are also covered by the present disclosure, without being explicitly mentioned in their respective formulation.

According to a first aspect of the disclosure, an imaging device for the scalable visual depiction of a region to be observed, in particular an operation region, is proposed. The device comprises an optical image capture apparatus for capturing an image of the region to be observed, optical and/or electronic scaling means associated with the image capture apparatus for scaling the captured image, and an adjustable focusing apparatus associated with the image capture apparatus, a robotic holding arm for moving the image capture apparatus relative to the region to be observed, wherein the holding arm is designed to provide a mechanical scaling function by adapting an axial distance between the image capture apparatus and the region to be observed, an input unit for capturing a user-side input command for scaling the captured image on a display unit, and a control apparatus for setting the scaling means of the image capture apparatus and the mechanical scaling function of the holding arm, as well as for setting the focusing apparatus. According to the disclosure, the control apparatus is designed to carry out an setting of the mechanical scaling function and the focusing apparatus by controlling or activating the holding arm and the focusing apparatus based only on the input command for scaling the captured image detected by the input unit, in particular a detected target value for the scaling.

According to a second aspect of the disclosure, a method for the scalable visual depiction of a region to be observed, in particular an operation region, is proposed. The method comprises at least the steps of: capturing an image of the region to be observed with an optical image capture apparatus for depiction on a display unit: initially setting the focus of the captured image by manually and/or automatically adjusting a focusing apparatus assigned to the image capture apparatus; capturing an input command for scaling the captured image by means of an input unit; scaling the captured image on the display unit by setting optical and/or electronic scaling means assigned to the image capture apparatus and setting a mechanical scaling function by moving the image capture apparatus along an optical axis of the image capture apparatus by means of a robotic holding arm; focusing the scaled captured image by adjusting the focusing apparatus.

In contrast to the prior art, according to the disclosure the setting of the mechanical scaling function is carried out by moving the holding arm and the focusing apparatus to track a focus or image sharpness of the captured image by controlling the focusing apparatus based only on the detected input command for scaling the captured image. This means that, in contrast to a control of holding arm movement and corresponding focus tracking known from the prior art, no further input variables, in particular no actual values for position or movement data of the holding arm, are detected as feedback values for a control of the mechanical scaling function and the focusing apparatus based thereon. In other words, the present control or activation of the mechanical scaling function and the focusing apparatus takes place without or independently of a detection of feedback values, in particular without detection of further input variables or values, in particular without detection of actual values for position or movement data of the holding arm that can be detected, for example, by corresponding sensors.

According to the disclosure, an improved imaging device and a corresponding method are hereby provided which enable a scalable image of an operation region with extended scaling or zoom functionality, and at the same time allow a simple and effective or efficient control of the system components, and in particular, adjustment of the holding arm and tracking of the image sharpness of the image. In so doing, a significantly faster and sufficiently accurate tracking of the focus adjustment based on the measured input variable can be made possible without the need for a control based on a determination of actual values, in particular for position or movement data of the holding arm.

This is independent of any possible internal control of the robotic holding arm or the focusing unit, which can each measure and compare their own actual and target positions during movement, also for safety reasons. However, such information is not used to control the other component.

In a preferred embodiment, the control apparatus is designed to control or activate the scaling means of the image capture apparatus, the mechanical scaling function of the holding arm, and the focusing apparatus based only on the input command for scaling the captured image detected by the input unit, and in particular without reading out or evaluating position or movement data of the robotic holding arm to provide a control variable. In this case, both the scaling means of the robotic holding arm and the focusing apparatus are actuated based only on the detected input command and thus not based on further control values, in particular, without detecting position or movement data of the robotic holding arm.

The control apparatus is preferably further designed to detect a target value for the scaling of the captured image or a desired change in the scaling of the captured image, and, based on the target value detected by the input unit, to actuate the scaling means of the image capture apparatus, the mechanical scaling function of the holding arm, and the focusing apparatus. In this case, respective target values for setting the scaling means, the mechanical scaling function and the focusing apparatus are preferably calculated based on the detected target value for a desired scaling of the captured image, wherein the control apparatus subsequently carries out a corresponding adjustment of the individual components on the basis of the calculated target values.

In a preferred embodiment, the mechanical scaling function of the holding arm is carried out preferably exclusively by a change in position of the image capture apparatus along its optical axis. In this case, an axial distance, i.e. a distance along the optical axis of the image capture apparatus, is changed between the image capture apparatus and the region to be observed.

The image capture apparatus is advantageously arranged on a holding element or manipulator, arranged preferably at the end of the robotic holding arm. The holding element or manipulator arranged at the end of the robotic holding arm is movable preferably based on a user input by means of an associated input device. The associated input device can be the device input unit, which can be selectively designed for the movable control of the robotic holding arm, in addition to actuating the mechanical scaling function. It is understood that a separate input unit can also be provided to actuate the holding arm.

In a preferred embodiment, the holding arm and/or the control apparatus is designed such that a change in the axial distance between the image capture apparatus and the region to be observed is possible only if the mechanical scaling function is provided. In other words, the holding arm and/or the control apparatus is designed such that a change in the axial distance between the image capture apparatus and the region to be observed is prevented or blocked by the device input unit or a separate input unit for actuating the holding arm, separately from the control command which provides the mechanical scaling function. The holding arm and/or the control apparatus are preferably designed such that a holding element arranged at the end or a manipulator of the robotic holding arm, on which the image capture apparatus is arranged, can be moved and/or rotated by the input unit in one or more dimensions around the region to be observed, wherein the axial distance between the image capture apparatus and the region to be observed is kept constant. The robotic holding arm and/or the control apparatus are preferably designed such that the image capture apparatus arranged on the holding arm can be moved, in particular on a spherical surface, for example, around the region to be observed, in particular an operation site or a trocar, while maintaining a constant axial distance between the image capture apparatus and the region to be observed.

The control apparatus of the device can be a central control apparatus which is connected at least to the input unit, the image capture apparatus and the robotic holding arm. Furthermore, the control apparatus can comprise at least one separate control unit, in particular for actuating the robotic holding arm, or can be selectively connected to this. The separate control unit is designed for preferably bidirectional data communication with the control apparatus of the device.

In a preferred embodiment, the scaling means are assigned a preferably internal memory and/or control unit for providing a minimum and maximum scaling factor as well as a current actual scaling factor to the control apparatus of the device. In this context, scaling factor is understood to mean an enlargement or reduction factor or zoom factor of the captured image.

The minimum and maximum scaling factors can be stored in the memory and/or control unit for the corresponding image capture apparatus. In this case, the minimum and maximum scaling factors can be dependent in particular on a corresponding optics of the image capture apparatus and/or a corresponding image sensor of the image capture apparatus. For example, the image capture apparatus can have an interchangeable optics, wherein different sets for a minimum and maximum scaling factor can be stored or are stored in the memory and/or control unit for different optics. The minimum and/or maximum limits can in particular correspond to a corresponding lower and/or upper limit for the reduction or enlargement by a zoom lens, which depend on the lenses used and their possible travel paths in the objective.

Furthermore, a different electronic or digital scaling factor can be stored or is stored depending on a particular image sensor and in particular depending on the image quality of the image sensor. In a further preferred embodiment, the values for a minimum and maximum scaling factor for the optical and/or electronic scaling means can also be configurable, in particular adaptable by user input.

The optical scaling means can in particular comprise a motor-actuated enlargement optics of the image capture apparatus for providing a selectively adjustable optical enlargement or zoom functionality of the image capture apparatus.

The electronic scaling means can in particular comprise a software-based enlargement and/or reduction and/or zoom functionality of the captured image, i.e., a so-called digital zoom.

The focusing apparatus of the device is preferably focusing optics assigned to the image capture apparatus, which can be adjusted selectively and preferably by a motor for adjusting the image sharpness of the captured image. In a preferred embodiment, the focusing apparatus is assigned a preferably internal memory and/or control unit for providing a minimum and maximum focus value or a focus setting as well as a current actual focus value or an actual focus setting to the control apparatus. The focus setting includes in particular a position of the focusing lens in an optics of the image capture apparatus. The corresponding values can be stored in the memory and/or control unit in a manner analogous to the above statements for a scaling factor for a corresponding image capture apparatus. In this case, a corresponding minimum and maximum focus value and/or a focus setting can be dependent in particular on a corresponding optics of the image capture apparatus and/or a corresponding image sensor of the image capture apparatus.

In a preferred embodiment, the preferably internal memory and/or control unit associated with the focusing apparatus is designed to provide a focus characteristic curve of the corresponding image capture apparatus to allow assigning a focus setting to each corresponding enlargement by the scaling means of the image capture apparatus and/or to a corresponding distance to the region to be observed, in particular for the mechanical scaling function. The focus characteristic curve preferably comprises a curve or characteristic curve from which a corresponding focusing lens position is expressed as a function of a corresponding distance to the region to be observed, for focusing the optics and/or the captured image. Each focus characteristic curve can be assigned to a corresponding image capture apparatus with an associated focusing lens in the memory and/or control unit, wherein the respective setting values for a focusing lens assigned to the image capture apparatus can be predefined and/or can be learned or are learned based on empirical measurement data with the corresponding image capture apparatus and the associated robotic holder arm.

In a preferred embodiment, the control apparatus is designed to compare a target value for the scaling detected by the input unit with a minimum and maximum scaling factor, as well as a provided actual scaling factor of the scaling means, i.e., a current zoom factor of the scaling means, and based thereon to effect a selective or combined actuation of the optical and/or electronic scaling means and of the robotic holding arm to provide the mechanical scaling function. In this case, the control apparatus can further be designed to carry out a preferred actuation of the optical and/or electronic scaling means and/or the mechanical scaling function based on the detected target value for the scaling and a comparison with a current actual scaling factor of the scaling means. In a particularly preferred embodiment, the control apparatus is designed firstly to actuate the optical and/or electronic scaling means until a minimum or maximum scaling factor of the scaling means is reached, and only then to carry out a further adjustment of the scaling by actuating the robotic holding arm, thus in accordance with the mechanical scaling function.

To provide the mechanical scaling function, the control apparatus is designed preferably to calculate a necessary change in the holding arm position, in particular a position change of the image capture apparatus arranged on the holding arm, along an optical axis of the image capture apparatus, based on the detected input command, in particular based on a target value for the scaling of the captured image. In this case, the control apparatus is preferably designed to output one or more target values for a movement, in particular a change in position of the robotic holding arm along the optical axis of the image capture apparatus for the change in position thereof. The corresponding position change can then be carried out by the control apparatus itself or a separate control unit of the robotic holding arm, to which the target values are provided or transmitted, actuating the robotic holding arm. Further preferably, the control apparatus is designed in such a way that the focusing apparatus is actuated, and in particular the position of the focusing lens of the image capture apparatus is adjusted, based on the calculated change in the holding arm position, in particular a change in the axial distance to the region to be observed. The focusing apparatus is preferably actuated by reading out a stored focus characteristic curve of the image capture apparatus, as described above.

The control apparatus can be designed in such a way that, when the captured image is scaled by adjusting the optical and/or electronic scaling means and/or the mechanical scaling function, a simultaneous and/or subsequent actuation of the focusing apparatus is determined for tracking the focus, preferably based on a stored focus characteristic curve of the corresponding image capture apparatus.

In a preferred embodiment, the input unit of the device is designed such that it enables a setpoint detection of a scaling factor, the setpoint detection being variable, in particular deflection-dependent. The input unit comprises preferably input means with multiple degrees of freedom, which are designed to detect a deflection in any direction. In particular, the input unit can comprise a 3D joystick which, in addition to detecting lateral input commands in a first plane of movement, enables detecting vertical input commands in a direction perpendicular to the first plane of movement. Furthermore, the 3D joystick can be designed to detect a deflection about a vertical axis of rotation and/or a tilt axis preferably arranged perpendicular thereto. A setpoint detection for the scaling factor can be carried out preferably by detecting a vertical input command, in particular in a first direction for reduction and in a second direction, opposite to the first, for enlargement. The input unit can be operated by a user, by a hand or a foot.

The input unit can further be designed to selectively actuate the image capture apparatus and/or the movable holding arm. In this case, the input unit can, for example, have a selective input function, for example a manually operable button or foot switch, by means of which a distinction can be made between a control of the holding arm by the input unit, in particular for changing the position and/or location of the holding arm, and a detection of a desired scaling factor or a detection of an adjustment of the current scaling factor.

In a preferred embodiment, the image capture apparatus is a stereo exoscope known per se. The stereo exoscope preferably has at least optical, controllable scaling means, in particular an optical zoom, as well as a motor-driven focus controller. The image capture apparatus can further comprise a distally arranged mirror unit for a 90° deflection of the viewing or capture direction. The mirror unit can optionally be designed to be movable or rotatable. The image capture apparatus can, for example, have two to four image sensors which are designed to capture a (stereo) image with a resolution of preferably 4K or higher.

In a further preferred embodiment, the image capture apparatus comprises an autofocus function which can be activated and/or actuated by the control apparatus. For example, the control apparatus can be designed to activate a preferably one-time, i.e., non-continuous, autofocus function of the image capture apparatus after completion of a holding arm movement. Since this actuation of the mechanical scaling function and/or the holding arm takes place without feedback of sensor data from a holding arm, the control apparatus is designed, for example, in such a way that, based on the respective calculated target values for a movement of the holding arm, a time estimate is made of how long the robotic holding arm requires to implement the desired or calculated movement, and based on this, the autofocus function is actuated after the previously calculated time interval has elapsed.

An autofocus function of the image capture apparatus can be implemented using means known per se; in particular, the autofocus function can comprise a contrast measurement, a phase comparison or an active distance measurement.

In a preferred embodiment of the method according to the disclosure, scaling and adjustment of the focusing apparatus or focus tracking of the captured image takes place by actuating the scaling means, the robotic holding arm and the focusing apparatus based only on the input command for scaling the captured image detected by the input unit, and in particular without reading out or evaluating position or movement data of the robotic holding arm. Scaling and focusing of the captured image can be done simultaneously or sequentially.

In a preferred embodiment of the method, based on a detected target value for scaling the captured image, in particular for a desired enlargement of the captured image, initially scaling is carried out by actuating the scaling means of the image capture apparatus; and only when a maximum scaling factor of the image capture apparatus is exceeded is scaling carried out by changing the axial distance between the image capture apparatus and the region to be observed, by changing the position of the holding arm. Furthermore, a control function can advantageously be carried out in such a way that, when a desired reduction in the captured image is desired, scaling is carried out firstly by changing the axial distance between the image capture apparatus and the region to be observed by changing the position of the holding arm, in particular by increasing the axial distance, and only when a predefined scaling factor is exceeded is the scaling means of the image capture apparatus actuated.

However, the reverse order is also possible in each case, so that for scaling with a desired enlargement, firstly mechanical scaling is carried out by the holding arm, for example if the control system detects that a desired target scaling is not possible by the scaling means of the image capture apparatus alone. Further scaling is then carried out using optical and/or electronic scaling means. The order is the same for reducing the size.

In a further preferred embodiment, actuating the focusing apparatus to change the holding arm position comprises at least the following further steps: determining an initial focus setting before executing a movement of the holding arm; calculating the necessary change in the holding arm position along the optical axis of the image capture apparatus to provide the detected target value for scaling the captured image; calculating a target value for the focus setting based on the calculated change in the holding arm position and on the basis of a focus characteristic curve stored for the image capture apparatus; actuating the focusing apparatus to set the determined target value.

### BRIEF DESCRIPTION OF DRAWINGS

Further advantages and details of the disclosure can be seen from the following description of preferred embodiments of the disclosure and from merely schematic drawings.

FIG. 1 is a schematic view of an embodiment of a device according to the disclosure;

FIG. 2 is a side view of an embodiment of an image capture apparatus according to the disclosure;

FIG. 3 is a block diagram of the device components; and

FIG. 4 is a schematic flow chart outlining the steps of a preferred embodiment for the scaling and focus tracking,

FIG. 5 is a schematic illustration of the application areas of optical scaling and mechanical scaling means; and

FIG. 6 is a schematic illustration of a focus characteristic curve;

Identical elements or elements with the same function are marked with the same reference numbers in the figures.

### DETAILED DESCRIPTION OF PREFERRED EMBODIMENTS

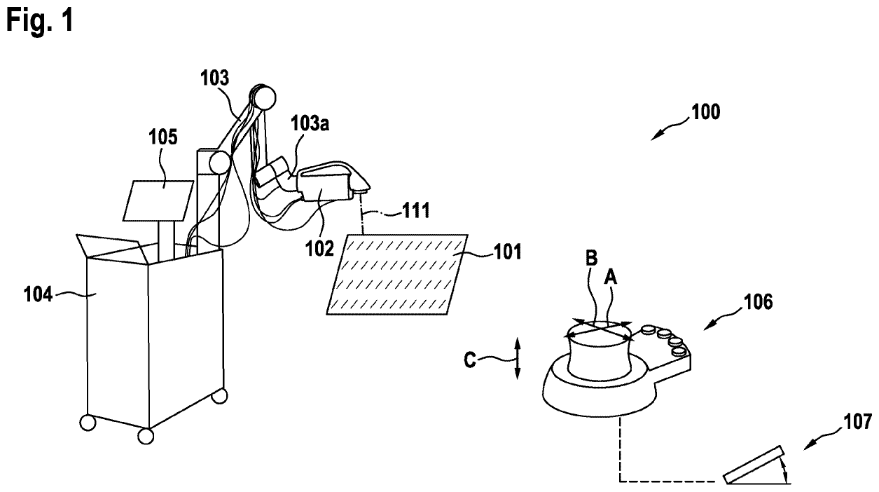

FIG. 1 is a schematic illustration of an embodiment of a device 100 according to the disclosure. The device is designed for the scalable visual depiction of a region 101 to be observed, in particular an operation region, shown here only schematically by way of example. The region 101 to be observed can be a surgical site.

The device 100 has an image capture apparatus 102, which is designed to capture an image, in particular a live video image. The image capture apparatus 102 can be arranged or is arranged, for example, at a distance of 20 to 50 cm above an area to be captured. The image capture apparatus 102 is arranged on an end portion or manipulator 103a of a robotic holding arm 103. The robotic holding arm 103 can be arranged on a stand element 104 such as a selectively movable carriage. Alternatively, ceiling or wall mounting in a treatment room is also possible. The image capture apparatus 102 is further connected to a display unit 105, on which the captured and selectively scalable image is output. The device 100 further comprises an input unit 106 and preferably a foot switch 107 associated therewith. The foot switch 107 can be designed to selectively switch the actuation of the robotic holding arm 103 or the image capture apparatus 102. The input unit 106 is designed preferably as a 3D joystick and preferably enables the detection of a vertical movement (double arrow C) in addition to the detection of lateral input commands (double arrows A, B). Furthermore, the input unit 106 can be designed to detect a rotational movement and/or a tilting movement of the 3D joystick (not shown). The input unit 106 is designed in particular to detect an input command for scaling or changing the scaling of the captured image shown on the display unit 105. In this case, the input unit is preferably designed to detect a deflection-dependent setpoint value of the scaling factor, in particular a deflection-dependent change of a current scaling factor. This means that a greater deflection of the 3D joystick along the double arrow C leads to a greater enlargement or reduction of the captured image on the display unit 105.

Scaling, i.e., a reduction or enlargement, of the captured image can be carried out by means of optical and/or electronic scaling means 108, 109 assigned region to be observed to the image capture apparatus 102 (see FIG. 3) and/or by means of a mechanical scaling function 110 provided by the holding arm (see FIG. 3), in which the robotic holding arm moves the image capture apparatus 102 held thereon relative to the region 101 to be observed along an optical axis 111 of the image capture apparatus 102 to adapt an axial distance between the image capture apparatus and the region to be observed (double arrow D).

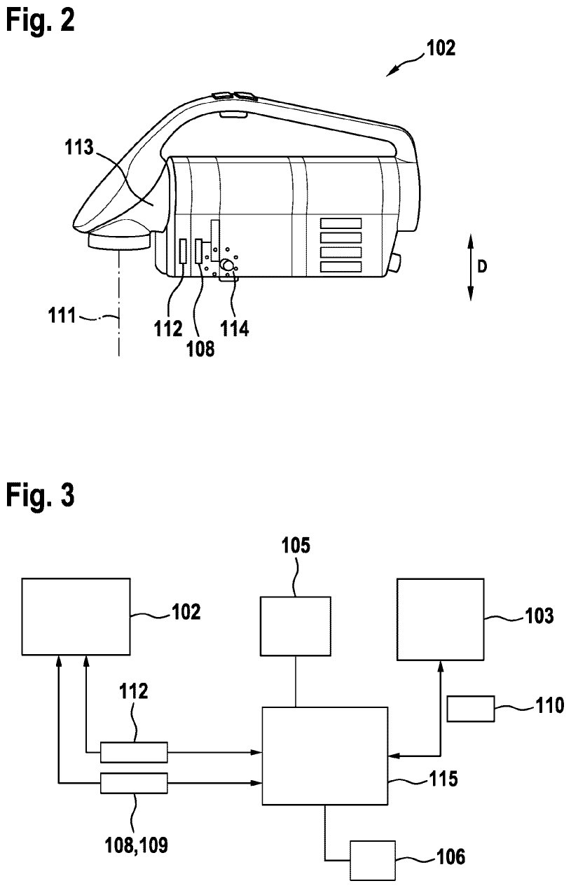

FIG. 2 is a side view of a preferred embodiment of the image capture apparatus 102. The image capture apparatus in this case is a known stereo exoscope. This has preferably motor-actuated optical scaling means, i.e., an optical zoom 108 assigned to or comprised by the optics of the exoscope, and a preferably motor-actuated focusing apparatus 112, i.e., a focusing lens assigned to or comprised by the optics of the exoscope. The image capture apparatus 102 further comprises a distally arranged mirror unit 113 for a 90° deflection of the viewing or detection direction. Furthermore, the image capture apparatus 102 can have a filter wheel 114 for receiving different, application-specific color filters. The image capture apparatus may additionally comprise an illumination unit (not shown) for illuminating the region to be observed.

FIG. 3 is a block diagram of the device components of an embodiment of the device according to FIG. 1. The device 100 comprises a control apparatus 115 which is connected to the image capture apparatus 102, to the robotic holding arm 103, and to the input unit 106. The control apparatus 115 may comprise a central control unit with a processor and associated memory device (not shown).

According to the disclosure, the control apparatus is designed such that it carries out the adjustment of the mechanical scaling function 110 and the focusing apparatus 112 by actuating the holding arm 103 and the focusing apparatus 112, without a control loop relying on sensor values of the holding arm 103, in particular without actual values for position or movement data of the holding arm 103. In particular, the control apparatus 115 is intended to control the holding arm 103 and the focusing apparatus 112 based only on an input command detected by the input unit 106 for scaling the captured image or for changing the scaling of the captured image. The control unit 115 is designed to actuate the mechanical scaling function 110 of the holding arm 103 by varying an axial distance d between the image capture apparatus 102 and the region 101 to be observed.

The control apparatus 115 is designed such that it can actuate an optical scaling function 108 of the image capture apparatus 102, in particular by adjusting the optics, and/or an electronic scaling function 109, in particular by displaying a subregion of the digital image area on the display unit 105. In this case, the control apparatus 115 is preferably designed to read out a corresponding actual value of the optical and/or electronic scaling means 108, 109, in particular a current enlargement or zoom factor. For this purpose, the optical and/or electronic scaling means 108, 109 can have an internal memory and/or control unit which, in addition to an actual value for a given scaling factor, stores the minimum and maximum scaling factors.

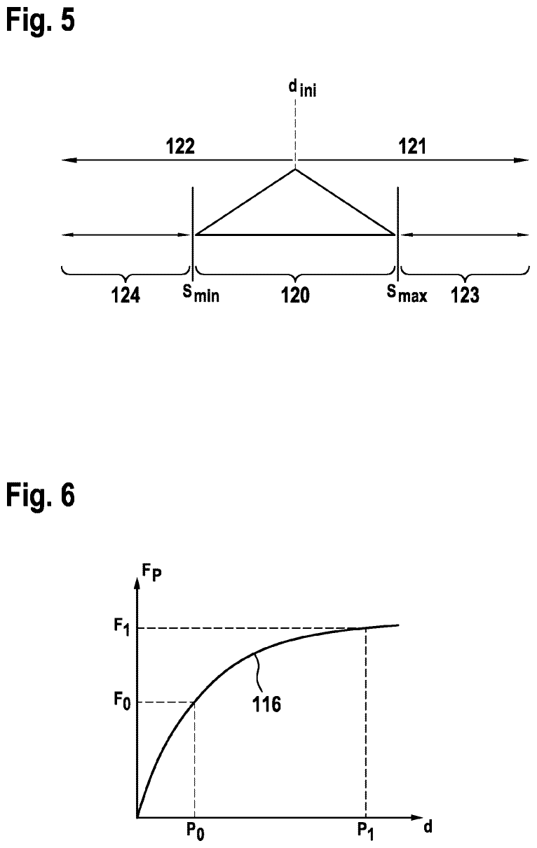

The control apparatus 115 is further designed for the selective or combined actuation of the scaling means 108, 109 and of the mechanical scaling function 110 in order to provide an extended scaling range or an extended overall zoom range (see also FIG. 5). The control apparatus 115 enables focus tracking of the captured image by preferably simultaneously or sequentially actuating the focusing apparatus 112. The focusing apparatus 112 preferably also has an internal memory and/or control unit, which can provide a current actual value of a focus setting of the focusing apparatus 112 to the control apparatus. Furthermore, each focus characteristic curve 116 for the corresponding image capture apparatus and/or optics or identification number of the image capture apparatus 102 is stored in the focusing apparatus 112 and/or in the control apparatus 115, indicating the relationship of a corresponding focus setting or a position of the focusing lens F_{P} in the optics of the image capture apparatus 102 to the corresponding distance d of the image capture apparatus 102 or the optics of the image capture apparatus 102 to the region 101 to be observed. In particular, the stored focus characteristic curve allows reading out the position of the focusing lens to which the corresponding focusing apparatus is to be set in order to achieve a focus of the captured image at a certain distance d between the detection optics and the region 101 to be observed.

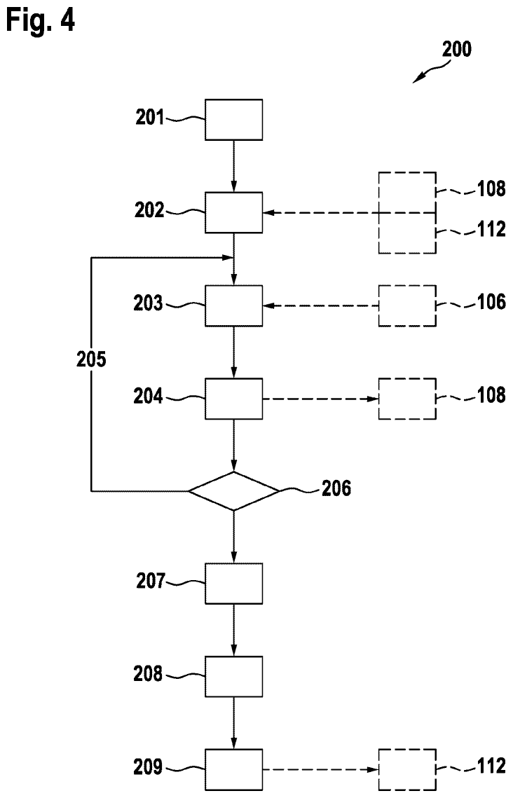

An embodiment of the steps for scaling and focus tracking with the device according to the disclosure is described below in connection with FIG. 4.

FIG. 4 shows a flow chart 200 for an actuation according to the disclosure of optical scaling means 108 of an image capture apparatus 102 and of the mechanical scaling function 110 for providing an expanded scaling, with tracking of a focus adjustment, for example for an enlargement of the captured image.

It is assumed that before using the device, a manual and/or electronic adjustment of the position of the image capture apparatus 102 with respect to the region 101 to be observed is carried out, in particular by a desired alignment of the robotic holding arm 103 and the image capture apparatus 102. The user can select a desired viewing angle and a desired distance d between image capture apparatus 102 and the region 101.

In a first step 201, an initial focus setting of the captured image is carried out by manual and/or electronic adjustment of a focusing apparatus 112 assigned to the image capture apparatus 102. Furthermore, an initial scaling or enlargement factor can additionally be set by manual and/or electronic adjustment, for example of the optical scaling means 108.

In step 202, the scaling means 108 and the focusing apparatus 112 are read out by the control apparatus 115 to provide actual values for a current scaling factor of the image capture apparatus 102 and a focus setting or a set position of the focusing lens of the optics of the image capture apparatus 102. In this way, the focal distance or the distance d between the image capture apparatus 102 and the viewing region 101 can be determined using the stored focus characteristic curve (position P₀, see FIG. 6).

The step 202 can be triggered, for example, by pressing a foot switch 107, wherein the foot switch 107 can serve as a trigger for a possibility of changing a scaling via input in the input unit and/or the 3D joystick 106. Alternatively, step 202 can also be triggered directly at the input unit 106.

In step 203, the input of a desired scaling factor or a desired change in the scaling factor is detected by means of the input unit 106. The input unit 106 can detect a target value for a desired enlargement, which is transmitted to the control apparatus 115.

In step 204, a new scaling or enlargement factor which is to be set by the device is calculated, based on the user input or the desired enlargement. A comparison is made with the detected actual scaling factor and a minimum and maximum value for the scaling factor of the scaling means 108. An enlargement by means of the scaling means 108 of the image capture apparatus 102 can take place until a stored maximum value of the scaling means 108 is reached, without the mechanical scaling function 110 being activated: see iteration loop 205.

If the desired scaling value exceeds the maximum value of the scaling means 108 (verification in step 206), a necessary distance change dl to the region 101 to be observed which is necessary to achieve the desired remaining enlargement is calculated in step 207. Based on this, a target value is then output in step 208 to actuate the robotic holding arm 103 and thus to change and in particular shorten the distance d along the optical axis 111 towards a position P₁ of the holding arm 103.

In step 209, the focusing apparatus 112 is actuated by the control apparatus 115 to track the image sharpness. Based on the input command for enlarging the captured image detected by the input unit 106, the focusing lens is adjusted using the stored focus characteristic curve. In particular, the control apparatus 115 determines the necessary adjustment of the focusing device and/or the position F₁ of the focusing lens in the optics of the image capture apparatus 102 based on the previously determined distance change d1, and thus, the new position P₁ (see FIG. 6).

If now, starting from the new position P₁, a reduction of the scaling factor is desired again and/or is detected by the input unit 106, the control apparatus 115 preferably actuates it in such a way that the mechanical scaling function 110 is initially made to reduce the scaling factor. In particular, the axial distance d is increased until the previously set distance change d1 is reached. The scaling means 108 of the image capture apparatus 102 are then actuated.

If a further reduction of the scaling factor is desired or is detected by the input unit 106, the scaling means 108 are actuated for the reduction until a stored minimum value of the scaling means is reached. Once the minimum value of the scaling means 108 is reached, the mechanical scaling function 110 is then actuated by increasing the axial distance d again, i.e., the image capture apparatus 102 is moved further away along the optical axis 111.

The actuation by the control apparatus 115 described above is explained again below with reference to the schematic illustration in FIG. 5, which shows a scaling range 120 of the exemplary optical scaling means 108 of the image capture apparatus 102. Starting from an initially determined distance dᵢₙᵢ between the image capture apparatus 102 and the region 101 to be observed, the optical scaling means 108 are initially actuated for enlargement (arrow 121) or reduction (arrow 122) until a maximum scaling factor Sₘₐₓ or a minimum scaling factor Sₘᵢₙ is reached. If the maximum scaling factor is exceeded or the minimum scaling factor is exceeded, the mechanical scaling function 110 is actuated, either to enlarge by shortening the axial distance d between the image capture apparatus 102 and the operation region 101 in the area 123 or to reduce the size by increasing the axial distance in the range 124.

Should the respective scaling factor be reset, i.e., from the regions outside the minimum and maximum scaling factors Sₘᵢₙ, Sₘₐₓ of the image capture apparatus 102 towards the range 120, the mechanical scaling function is actuated firstly to reduce or increase the scaling factor until a distance is reached which lies within the range 120 in which the optical scaling means 108 can be used.

The actuation described above may depend on the activation of a foot switch. If, for example, the foot switch has been released after a reduction (arrow 122) into the range 124, then, as opposed to the control described above and, for example, in the case of a (further) increase in a scaling factor from the range 124, the optical scaling means 108 can initially be actuated immediately; and only when a maximum possible scaling factor of the optical scaling means 108 is reached, then the mechanical scaling function 110 can be actuated for further enlargement.

1. An imaging device for the scalable visual depiction of a region to be observed, in particular an operation region, the imaging device comprising:
an optical image capture apparatus for capturing an image of the region to be observed
optical and/or electronic scaling means associated with the image capture apparatus for scaling the captured image, and an adjustable focusing apparatus associated with the image capture apparatus;
a robotic holding arm for moving the image capture apparatus relative to the region to be observed, wherein the holding arm is designed to provide a mechanical scaling function through adaptation of an axial distance (d) between the image capture apparatus and the region to be observed;
an input unit for capturing a user-side input command for scaling the captured image on a display unit;
a control apparatus for setting the scaling means of the image capture apparatus and the mechanical scaling function of the holding arm, as well as for setting the focusing apparatus;
characterized in that the control apparatus is designed to carry out the setting of the mechanical scaling function and the focusing apparatus by controlling the holding arm and the focusing apparatus, based only on the input command detected by the input unit for scaling the captured image.

2. The imaging device according to claim 1, wherein the control apparatus is designed to actuate the scaling means, the mechanical scaling function of the holding arm, and the focusing apparatus based only on the input command for scaling the captured image detected by the input unit, and in particular without reading out or evaluating position or movement data of the robotic holding arm.

3. The imaging device according to claim 1, wherein the input unit is designed to detect a target value for the scaling of the captured image, and in that the control apparatus is designed to actuate the scaling means of the image capture apparatus, the mechanical scaling function of the holding arm, and the focusing apparatus based on the target value detected by the input unit.

4. The imaging device according to claim 1, wherein the mechanical scaling function of the holding arm is carried out by a change in position of the image capture apparatus along its optical axis.

5. The imaging device according to claim 1, wherein the scaling means are assigned a preferably internal memory and/or control unit for providing a minimum and maximum scaling factor as well as a current actual scaling factor to the control apparatus.

6. The imaging device according to claim 1, wherein the focusing apparatus is assigned a preferably internal memory and/or control unit for providing a minimum and maximum focus value/focus adjustment as well as a current actual focus value/focus adjustment to the control apparatus.

7. The imaging device according to claim 1, wherein the control apparatus and/or a preferably internal memory and/or control unit assigned to the focusing apparatus is designed to provide a focus characteristic curve of the image capture apparatus for the purpose of assigning a corresponding focus adjustment to a corresponding distance (d) to the region to be observed.

8. The imaging device according to claim 1, wherein the control apparatus is designed to compare a target value for the scaling detected by the input unit with a minimum and maximum scaling factor, as well as the actual scaling factor of the scaling means and, based thereon, to actuate a selective or combined control of the optical and/or electronic scaling means and of the robotic holding arm for providing the mechanical scaling function.

9. The imaging device according to claim 1, wherein the control apparatus is designed firstly to actuate the optical and/or electronic scaling means until a minimum or maximum scaling factor of the scaling means is reached, and only then to carry out a further adjustment of the scaling by actuating the robotic holding arm.

10. The imaging device according to claim 1, wherein the control apparatus is designed to calculate a necessary change in the holding arm position, in particular a position change of the image capture apparatus arranged on the holding arm, along an optical axis of the image capture apparatus based on a target value for the scaling of the captured image, and to actuate the focusing apparatus based on this change in the holding arm position, in particular a change in the axial distance (d) to the region to be observed.

11. The imagined device according to claim 10, wherein the control apparatus is designed to determine a simultaneous and/or subsequent actuation of the focusing apparatus for tracking the focus based on a stored focus characteristic curve of the corresponding image capture apparatus when scaling the captured image.

12. The imaging device according to claim 1, wherein the input unit enables detecting a variable and particularly deflection-dependent setpoint value of a scaling factor, and is optionally designed for the selective actuation of the image capture apparatus and/or the movable holding arm.

13. The imaging device according to claim 1, wherein the image capture apparatus includes a stereo exoscope with preferably at least optical scaling means and a motor-driven focusing apparatus.

14. The imaging device according to claim 1, wherein the movable robotic holding arm has its own control unit which is designed for bidirectional data communication with the control apparatus of the device.

15. The imaging device according to claim 1, wherein the control apparatus is designed to activate a preferably one-time, i.e., non-continuous, autofocus function of the image capture apparatus upon termination of a holding arm movement.

16. A method for the scalable visual depiction of a region to be observed, in particular an operation region, the method comprising at least the following steps:
capturing an image of the region to be observed with an optical image capture apparatus for depiction on a display unit;
initially setting the focus of the captured image by manual and/or automatic adjustment of a focusing apparatus assigned to the image capture apparatus;
detecting an input command for scaling the captured image, by means of an input unit;
scaling the captured image on the display unit by setting optical and/or electronic scaling means (108, 109) assigned to the image capture apparatus and setting a mechanical scaling function by moving the image capture apparatus along an optical axis of the image capture apparatus by means of a robotic holding arm;
focusing the scaled captured image by adjusting the focusing apparatus; and
wherein the mechanical scaling function and the focusing apparatus are adjusted, by controlling the holding arm and the focusing apparatus, based only on the input command for scaling the captured image detected by the input unit.

17. The method according to claim 16, wherein for scaling the captured image and adjusting the focusing apparatus, the scaling means (108, 109), the robotic holding arm and the focusing apparatus are actuated based only on the input command for scaling the captured image detected by the input unit, and in particular without reading out or evaluating position or movement data of the robotic holding arm.

18. The method according to claim 16, wherein the scaling the captured image and focusing the captured image are preferably performed simultaneously or sequentially.

19. The method according to claim 16, wherein based on a detected target value for scaling the captured image, scaling is carried out firstly by actuating the scaling means of the image capture apparatus, and only when a maximum scaling factor of the image capture apparatus is exceeded is scaling carried out by changing the axial distance (d) between the image capture apparatus and the region to be observed, by changing the position of the holding arm.

20. The method according to claim 16, wherein an actuation of the focusing apparatus during a change in the holding arm position comprises at least the following further steps:
determining an initial focus adjustment before executing a movement of the holding arm;
calculating the necessary change in the holding arm position along the optical axis of the image capture apparatus to provide the detected target value for scaling the captured image;
calculating a target value for the focus adjustment based on the calculated change in the holding arm position and on the basis of a focus characteristic curve stored for the image capture apparatus; and
actuating the focusing apparatus to set the determined target value.
